(19) United States
(12) Patent Application Publication
(10) Pub. No.: US 2004/0197412 A1
(43) Pub. Date: Oct. 7, 2004

(54) Process for preparing pharmaceutical formulations using supercritical fluids

(75) Inventors: Satish Pejaver, Bridgewater, NJ (US); Navneet Puri, Bridgewater, NJ (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: BAXTER INTERNATIONAL, INC., Deerfield, IL

(21) Appl. No.: 10/743,448

(22) Filed: Dec. 19, 2003

Related U.S. Application Data
(60) Provisional application No. 60/435,054, filed on Dec. 19, 2002.

(51) Int. Cl. A61K 31/43; A61K 9/14

(52) U.S. Cl. 424/489; 514/192

(57) ABSTRACT

The invention is directed to a process for preparing a pharmaceutical formulation containing two or more active pharmaceutical ingredients comprising: (a) combining two or more active pharmaceutical ingredients with a supercritical fluid to form a supercritical fluid solution; and (b) separating the active ingredients from the supercritical solution to yield a dry powder precipitate. Preferably, the pharmaceutical formulation prepared according to the invention contains a combination of two anti-infective agents. The invention is further directed to a process for preparing a pharmaceutical formulation containing two or more active pharmaceutical ingredients comprising: (a) combining two or more active ingredients with a cosolvent to form a solution; (b) mixing the solution with a supercritical fluid; and (c) recovering the precipitate in a dry powder form.

### CROSS-REFERENCE TO RELATED PATENT APPLICATIONS

This patent application claims the benefit of U.S. Provisional Patent Application No. 60/435,054, filed Dec. 19, 2002.

### FIELD OF THE INVENTION

This invention pertains to a process for combining two or more active pharmaceutical ingredients including, for example, anti-infective agents, using a supercritical fluid to obtain a blended, dry powder pharmaceutical formulation.

### BACKGROUND OF THE INVENTION

Pharmaceuticals containing a combination of two or more active pharmaceutical ingredients, especially anti-infective agents, are commercially available in a dry powder form. Anti-infective agents as well as many other active agents are not stable for extended periods of time in an aqueous solution which requires the preparation of such actives as a solid powder.

Combination anti-infectives are typically produced by milling of the active agents and excipients and blending the dry solid components to form the finished drug product. However, the use of milling and blending techniques has several significant limitations. Most significantly, the mechanical equipment used to accomplish the milling and blending operations is in direct contact with the drug product components which can result in contamination from pyrogens and/or particular matter. Such contaminants destroy the sterility required for pharmaceutical products that are administered parenterally. Other drawbacks include, for example, the need for specialized ventilation equipment to collect dust produced during milling, the difficulty in obtaining blend uniformity, and the degradation of the active ingredients and excipients caused by high shear milling. Moreover, the potential segregation of the components of the blended powder during its transfer from blender to the filling line and during vial filling may eventually lead to content non-uniformity in the final blended drug product.

An alternative to the use of traditional milling and blending procedures to produce combination drug products is spray drying. The spray drying process involves the dissolution of active agents in a suitable cosolvent (which may be a single solvent or two or more solvents combined together) following by spraying of the solution in a heated chamber. However, spray drying has several significant limitations. Stability issues exist with the solution or dispersion of the active agents formed before spraying. In addition, the high temperatures used during the process can cause degradation. Spray drying also gives low yields of the final product and often requires the use of a secondary drying step to ensure removal of cosolvent from the powder.

Thus, there remains a need for an efficient process for producing sterile combination pharmaceutical drug products in a powder form that exhibit good blend uniformity.

The invention provides such a process for preparing sterile pharmaceutical formulations in a uniform, dry powder form that contain two or more active pharmaceutical ingredients. These and other advantages of the invention, as well as additional inventive features, will be apparent from the description of the invention provided herein.

### BRIEF SUMMARY OF THE INVENTION

The invention provides a process for preparing a pharmaceutical formulation containing two or more active pharmaceutical ingredients by (a) combining two or more active ingredients with a supercritical fluid, and (b) separating the active ingredients from the supercritical fluid to yield a dry powder precipitate containing the active ingredients. The invention further provides a supercritical fluid solution comprising a supercritical fluid and two or more active pharmaceutical ingredients.

The invention further relates to a process for preparing a pharmaceutical formulation containing a combination of two anti-infective agents comprising:

(a) combining two anti-infective agents with supercritical carbon dioxide to form a supercritical carbon dioxide solution;

(b) spraying the supercritical carbon dioxide solution through a nozzle; and

(c) recovering the precipitate in a dry powder form containing the combination of anti-infective agents.

In another embodiment, the invention is directed to a process for preparing a pharmaceutical formulation containing two or more active pharmaceutical ingredients comprising:

(a) combining two or more active ingredients with a cosolvent to form a solution;

(b) mixing the solution with a supercritical fluid; and

(c) recovering the precipitate in a dry powder form.

The invention further includes a process for preparing a pharmaceutical formulation containing a combination of two anti-infective agents comprising:

(a) combining two anti-infective agents with a cosolvent to form a solution;

(b) mixing the solution with supercritical carbon dioxide; and

(c) recovering the precipitate in a dry powder form containing the combination of anti-infective agents.

### BRIEF DESCRIPTION OF THE DRAWINGS

FIG. 1 is a schematic of the apparatus for the recrystallization of pharmaceutical formulations containing two or more active pharmaceutical ingredients using the RESS technique.

FIG. 2 is a schematic of the apparatus for the recrystallization of pharmaceutical formulations containing two or more active pharmaceutical ingredients using the SAS technique.

FIG. 3 is a schematic of the apparatus for the recrystallization of pharmaceutical formulations containing two or more active pharmaceutical ingredients using the GAS technique.

### DETAILED DESCRIPTION OF THE INVENTION

The present invention is directed to a process for preparing pharmaceutical formulations containing two or more active pharmaceutical ingredients using supercritical fluid technology.

A “supercritical fluid” is a fluid at or above its critical pressure (P_{c}) and critical temperature (T_{c}) simultaneously. Thus, a fluid above its critical pressure and at its critical temperature is in a supercritical state. A fluid at its critical pressure and above its critical temperature is also supercritical.

As used herein, supercritical fluids also encompass both near supercritical fluids and subcritical fluids. A “near supercritical fluid” is above but close to its critical pressure (P_{c}) and critical temperature (T_{c}) simultaneously. A “subcritical fluid” is above its critical pressure (P_{c}) and close to its critical temperature (T_{c}).

Any suitable supercritical fluid may be used in the process of the present invention. The supercritical fluid should be compatible with the active agents that are dissolved in or contacted with the supercritical fluid in the recrystallization processes detailed herein.

Typical supercritical fluids and their critical properties (i.e., critical temperature, critical pressure, and critical density) are listed in Table 1.

TABLE 1

| Fluid | T_{c} (° C.) | P_{c} (MPa) | ρ_{c} (g/cm³) |
|---|---|---|---|
| ethylene | 9.3 | 5.04 | 0.22 |
| xenon | 16.6 | 5.84 | 0.12 |
| carbon dioxide | 31.1 | 7.38 | 0.47 |
| ethane | 32.2 | 4.88 | 0.20 |
| nitrous oxide | 36.5 | 7.17 | 0.45 |
| propane | 96.7 | 4.25 | 0.22 |
| ammonia | 132.5 | 11.28 | 0.24 |
| n-butane | 152.1 | 3.80 | 0.23 |
| n-pentane | 196.5 | 3.37 | 0.24 |
| isopropanol | 235.2 | 4.76 | 0.27 |
| methanol | 239.5 | 8.10 | 0.27 |
| toluene | 318.6 | 4.11 | 0.29 |
| water | 374.2 | 22.05 | 0.32 |

Carbon dioxide is preferably utilized used as the supercritical fluid for producing pharmaceutical formulations containing two or more active agents according to the present invention. The use of supercritical carbon dioxide in pharmaceutical processing is further described in Subramaniam et al., J. Pharm. Sci. 1997: 86, 8, which is incorporated herein by reference.

Any combination of two or more active pharmaceutical ingredients may be used in the present invention. Preferably, two or more anti-infectives are combined in pharmaceutical formulations of the present invention. Some examples of anti-infectives suitable for use including macrolide antibiotics such as clarithromycin, erythromycin, and azithromycin, anthracycline antibiotics such as doxorubicin and daunorubicin, camptothecin and its analogs such as topotecan and irenotecan, and quinolone antibiotics such as ciprofloxacin, ofloxacin, levofloxacin, clinafloxacin, and moxifloxacin. Cephalosporins may also be used such as, for example, cefotaxime, ceftriaxone, ceftazidime, and cefepime.

Other suitable anti-infective agents include β-lactam antibiotics (e.g., cefotetan, aztreonam), penicillins (e.g., amoxicillin, piperacillin), aminoglycosides (e.g., streptomycin), and sulfonamides (e.g., trimethoprim/sulfamethoxazole). Further anti-infective agents and classes thereof that may be used include, without limitation, carbapenems, bacitracin, gramicidin, mupirocin, chloramphenicol, thiamphenicol, fusidate sodium, lincomycin, clindamycin, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, fluoroquinolones, isoniazid, ethambutol, pyrazinamide, para-aminosalicylic acid (PAS), cycloserine, capreomycin, ethionamide, prothionamide, thiacetazone and viomycin. Specific anti-infectives that are suitable include, without limitation, amikacin, netilmicin, fosfomycin, gentamicin, and teicoplanin.

Most preferably, pharmaceutical drug products containing two anti-infective active ingredients are produced according to the invention. The following combinations of anti-infective agents are preferably used: ampicillin sodium/sulbactam sodium (marketed under the brand name Unasyn® by Pfizer); ticarcillin disodium/clavulanate potassium (marketed under the brand name Timentin® by GlaxoSmithKline); quinupristin/dalfopristin (marketed under the brand name Synercid® by Aventis); piperacillin sodium/tazobactam sodium (marketed under the brand name Zosyn® by Lederle Pharmaceutical); and, imipenem/cilastatin (marketed under the brand name Primaxin® by Merck).

Other types of active pharmaceutical ingredients that may be combined according to the present invention include the following classes of drugs: anxiolytic (e.g., diazepam), antidepressant (e.g., fluoxetine), anesthetic (e.g., midazolam), antiviral (e.g., ganciclovir), protease inhibitor (e.g., saquinavir), chemotherapeutic (e.g., mesna, paclitaxel, cisplatin), anti-inflammatory (e.g., naproxen, ketorolac), antimalarial (e.g., mefloquine), antihypertensive (e.g., enalapril, lisinopril), antiseborheic (e.g., isotretinoin), calcium channel blocker (e.g., diltiazem, nifedipine), lipase inhibitor (e.g., orlistat), antiparkinson (e.g., tolcapone), antiarthritic (e.g., mycophenolate mofetil), and thrombolytic agent (e.g., streptokinase).

Any suitable salts of active pharmaceutical ingredients may be used including, for example, sodium, hydrochloride, potassium, mesylate, axetil, phosphate, succinate, maleate. Alternatively, the free acid form of active agents may be used.

Conventional processes using supercritical fluids for producing pharmaceutical particles may be used. Examples of preferred supercritical processing techniques for recrystallizing pharmaceuticals include Rapid Expansion from Supercritical Solutions (RESS), Supercritical Anti-Solvent (SAS), and Gas Antisolvent (GAS).

In the RESS process, the two or more active pharmaceutical ingredients are dissolved in a supercritical fluid, preferably carbon dioxide, to form a homogenous solution. Other excipients may optionally be added to the supercritical fluid. The active agents and optional excipients may be added to the supercritical fluid simultaneously or other suitable order. The resulting solution is then passed through an orifice or nozzle into a chamber. Preferably, the pressure in the chamber is atmospheric. By spraying the homogenous solution through an orifice or nozzle, the solution is depressurized rapidly resulting in the vaporization of the carbon dioxide or other supercritical fluid. The active agents and optional excipients are recrystallized as a uniform mixture in dry powder form.

RESS can be used if the active pharmaceutical ingredients to be precipitated are soluble in the supercritical fluid, such as supercritical carbon dioxide. If the active agents are not readily soluble in the supercritical fluid, the active agents may be first dissolved in a cosolvent system and then added to the supercritical fluid. The cosolvent may be a single solvent or two or more solvents combined together. Alternatively, the cosolvent may be added to the supercritical fluid initially followed by the addition of the active agents to the mixture of the supercritical fluid and cosolvent. When a cosolvent is required, the cosolvent used generally has a higher dielectric constant than the supercritical fluid (e.g., supercritical carbon dioxide), but is miscible in the supercritical fluid. Examples of suitable solvents and cosolvents include acetone, methanol, ethanol, propanol, butanol, tetrahydrorfuran, methylene chloride, chloroform, toluene, dimethylsufloxide, N,N-dimethylformamide, cyclohexanone, butrylactone, water, and combinations thereof. Other suitable solvents include those compounds known in the art in which the active pharmaceutical ingredients to be blended can be dissolved.

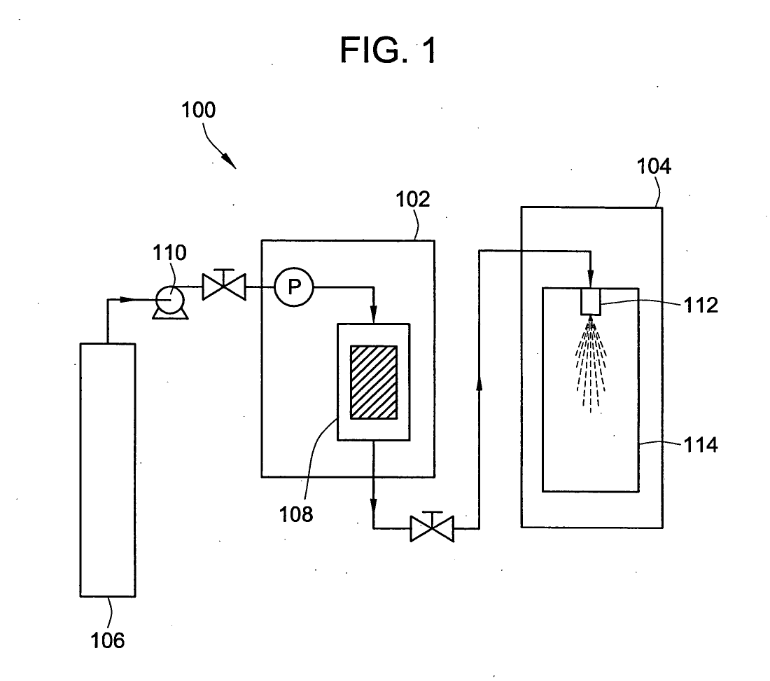

A typical flow diagram of a RESS process for recrystallization using carbon dioxide as the supercritical fluid is shown in FIG. 1. The RESS apparatus 100 generally includes an extraction unit 102 and precipitation unit 104. Carbon dioxide is transferred from storage tank 106 to high-pressure vessel 108, optionally using pump 110. The temperature and pressure in high-pressure vessel 108 are maintained such that the carbon dioxide exists in a supercritical state. The active pharmaceutical ingredients are then added to high-pressure vessel 108 to form a homogenous solution of the carbon dioxide in which the active agents are dissolved. Alternatively, the active pharmaceutical ingredients may be added to the high-pressure vessel 108 initially followed by the addition of supercritical carbon dioxide to form a homogeneous solution. The homogenous solution is sprayed through nozzle 112 into vessel 114, preferably under atmospheric pressure conditions. Alternatively, pressures greater than atmospheric pressure may be used. The supercritical carbon dioxide is vaporized and the active agents precipitate from the solution in the form of a dry powder. The carbon dioxide may either be collected for possible reuse or discarded. The solid precipitate is collected from vessel 114 for further processing.

The upstream and downstream temperatures and pressures in the RESS process may be modified to obtain the desired morphology of the precipitated drug product. In addition, the shape of the nozzle employed may be altered to transition between fibers and particles. A smaller length-to-nozzle diameter ratio (L/D) typically results in the formation of particles.

Another suitable process for recrystallization according to the present invention is the SAS process. The SAS technique is well-suited for precipitation of active agents that are only slightly soluble in the supercritical fluid of interest, such as supercritical carbon dioxide.

In the SAS process, the active pharmaceutical ingredients and optional excipients are dissolved in a cosolvent. The cosolvent may be any suitable liquid containing one or more solvents in which the active agents are dissolved. The cosolvent is also miscible in the supercritical fluid. Examples of cosolvents suitable for use in the SAS method include those cosolvents discussed herein that may be used in the RESS process as well as other cosolvents in which the active agents can be dissolved.

The solution containing the active agents is then mixed with a supercritical fluid (e.g., supercritical carbon dioxide). Preferably, mixing is carried out by spraying the solution through a nozzle into a chamber filled with the supercritical fluid. The supercritical fluid acts as an anti-solvent to extract out the cosolvent. The active agents and optional excipients form a precipitate upon contact with the supercritical fluid which is recovered. The precipitate from the SAS process is a uniformly mixed dry powder containing the active pharmaceutical ingredients and any optional excipients.

The supercritical fluid may optionally be mixed with one or more cosolvents prior to the addition of the solution containing the active agents.

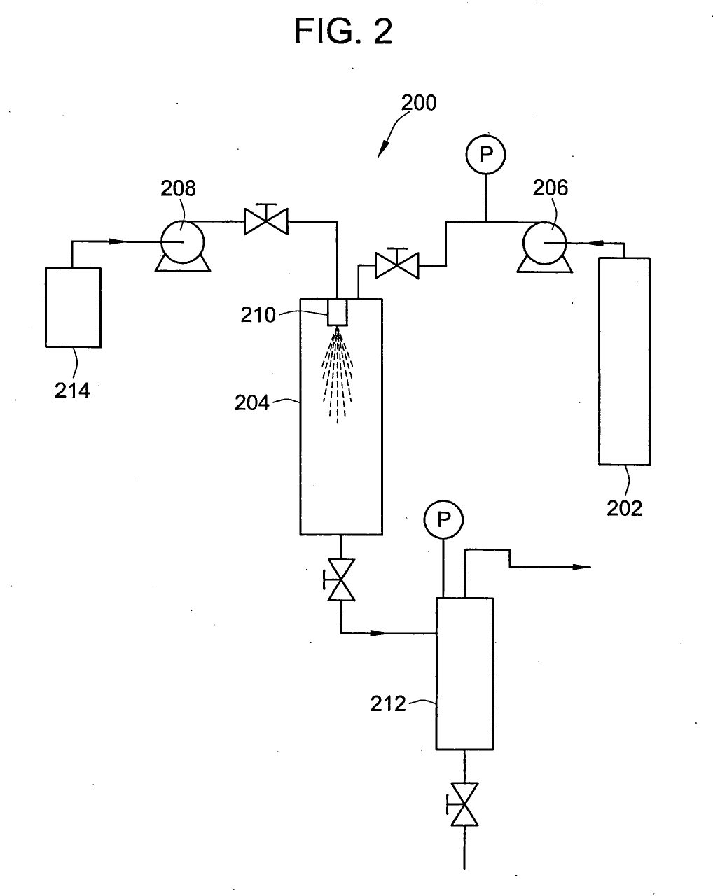

A typical flow diagram of a SAS process for recrystallization using supercritical carbon dioxide is shown in FIG. 2. In the SAS apparatus 200, the active pharmaceutical ingredients are dissolved in a cosolvent system in vessel 214. Excipients may optionally be dissolved in the cosolvent along with the active agents. Carbon dioxide is transferred from vessel 202 to high-pressure vessel 204, optionally using pump 206, wherein carbon dioxide is maintained in a supercritical state. The cosolvent solution containing the active agents is transferred from vessel 214, optionally using pump 208, and sprayed through nozzle 210 into high-pressure vessel 204. The precipitate containing a powder blend of active agents is recovered from high-pressure vessel 204 for further processing. The resulting mixture of the cosolvent system and supercritical carbon dioxide is then transferred to low-pressure tank 212 for recovery of the cosolvent and carbon dioxide and reuse of these process streams.

In the GAS process, supercritical carbon dioxide is rapidly added to a solution of the desired active pharmaceutical ingredients dissolved in an organic cosolvent. The supercritical carbon dioxide and organic solvent are miscible whereas the solid active agents have limited solubility in carbon dioxide. Thus, the carbon dioxide acts as an antisolvent to precipitate solid crystals of the active agents.

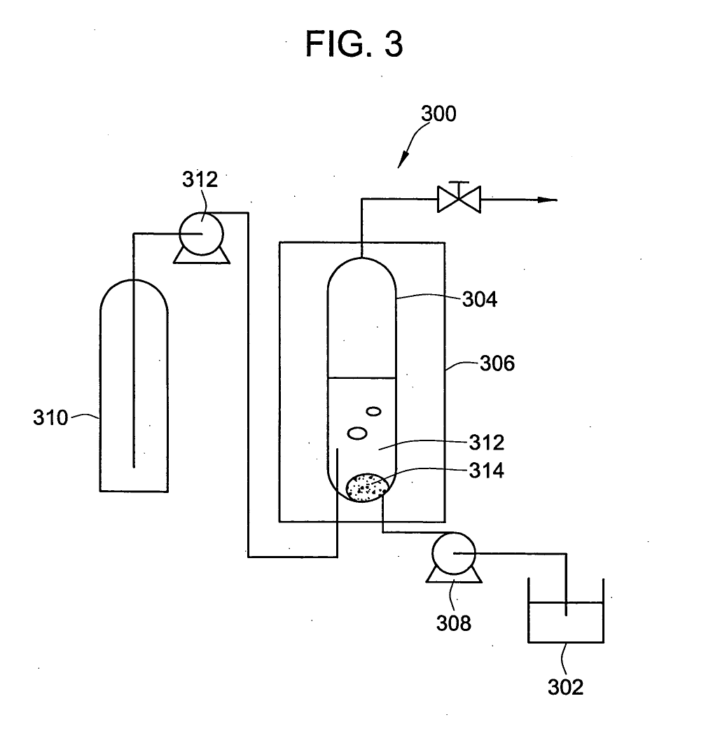

A typical flow diagram of a GAS process for recrystallization using supercritical carbon dioxide is shown in FIG. 3. In the GAS apparatus 300, the active pharmaceutical ingredients are dissolved in an organic cosolvent in vessel 302. Excipients may optionally be dissolved in the cosolvent along with the active agents. The solution in which the active agents are dissolved is transferred to a vessel 304 in the precipitator 306 using pump 308. Carbon dioxide stored in a supercritical state in vessel 310 is rapidly transferred to vessel 304 using pump 312. Alternatively, carbon dioxide may be stored as either a gas or liquid well below its critical temperature and critical pressure and then rendered supercritical before combining the carbon dioxide with the dissolved active agents. Upon contact with the supercritical carbon dioxide, the dissolved active agents in the solution 312 in vessel 304 are crystallized as particles 314 containing a blend of the active agents and optional excipients. The particles are recovered for further processing to yield a suitable pharmaceutical formulation.

The supercritical fluid may optionally be mixed with one or more cosolvents prior to the addition of the solution containing the active agents.

Other suitable processes known to those persons of ordinary skill in the art that involve a supercritical fluid, preferably supercritical carbon dioxide, may be used to recrystallize combinations of active pharmaceutical ingredients in a dry powder form.

The uniform blend of active pharmaceutical agents recrystallized using a supercritical fluid according to the process of the present invention is a dry powder. The precipitated powder typically contains about 10% or less (by weight) of the solvent in which the active agents are dissolved prior to crystallization. Preferably, the dry powder contains 5% or less solvent (by weight) and, most preferably, 2% or less (by weight) solvent.

The pharmaceutical formulations produced according to the present invention may optionally contain pharmaceutically acceptable excipients such as, for example, carriers, additives, and diluents. Pharmaceutical formulations for parenteral administration may contain, for example, alkylene glycols such as propylene glycol, polyalkylene glycols such as polyethylene glycol, oils of vegetable origin, hydrogenated naphthalenes, acid or basic buffers, and the like.

Other examples of suitable excipients for pharmaceutical dosage forms prepared by the present invention include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. Pharmaceutical formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical active ingredients used in the present invention may be premixed with one or more pharmaceutically acceptable excipients before the active agents are contacted with a supercritical fluid according to the inventive processes. When premixed with the active agents, the excipients must be compatible with the cosolvent systems and supercritical fluids that are employed.

Alternatively, a uniform blend of two or more active pharmaceutical ingredients obtained by recrystallization using a supercritical fluid may be mixed with one or more pharmaceutically acceptable excipients to produce a pharmaceutical formulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A process for preparing a pharmaceutical formulation containing two or more active pharmaceutical ingredients comprising:
(a) combining two or more active pharmaceutical ingredients with a supercritical fluid to form a supercritical fluid solution; and
(b) separating the active ingredients from the supercritical fluid solution to yield a dry powder precipitate.

2. The process according to claim 1, wherein the supercritical fluid is carbon dioxide.

3. The process according to claim 2, wherein at least one of the active pharmaceutical ingredients is an anti-infective agent.

4. The process according to claim 3, wherein the anti-infective agent is selected from the group consisting of macrolide antibiotics, anthracycline antibiotics, quinolone antibiotics, cephalosporins, β-lactam antibiotics, penicillins, aminoglycosides, and sulfonamides.

5. The process according to claim 2, wherein two or more of the active pharmaceutical ingredients are a combination of anti-infective agents.

6. The process according to claim 5, wherein the combination of anti-infective active ingredients are selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

7. The process according to claim 2, wherein the active ingredients are separated from the supercritical carbon dioxide solution by spraying the solution through a nozzle and recovering the precipitate.

8. The process according to claim 7, wherein the active pharmaceutical ingredients are mixed with a cosolvent prior to step (a).

9. The process according to claim 7, wherein the supercritical fluid is mixed with a cosolvent prior to step (a).

10. A process of preparing a pharmaceutical formulation containing a combination of two anti-infective agents comprising:
(a) combining two anti-infectives with supercritical carbon dioxide to form a supercritical carbon dioxide solution;
(b) spraying the supercritical carbon dioxide solution through a nozzle; and
(c) recovering the precipitate in a dry powder form containing the combination of anti-infective agents.

11. The process according to claim 10, wherein the combination of anti-infective agents is selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

12. A process for preparing a pharmaceutical formulation containing two or more active pharmaceutical ingredients comprising:
(a) combining two or more active ingredients with a cosolvent to form a solution;
(b) mixing the solution with a supercritical fluid; and
(c) recovering the precipitate in a dry powder form.

13. The process according to claim 12, wherein the supercritical fluid is carbon dioxide.

14. The process according to claim 13, wherein at least one of the active pharmaceutical ingredients is an anti-infective agent.

15. The process according to claim 14, wherein the anti-infective agent is selected from the group consisting of macrolide antibiotics, anthracycline antibiotics, quinolone antibiotics, cephalosporins, β-lactam antibiotics, penicillins, aminoglycosides, and sulfonamides.

16. The process according to claim 13, wherein two or more of the active pharmaceutical ingredients are a combination of anti-infective agents.

17. The process according to claim 16, wherein the combination of anti-infective active ingredients are selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

18. The process according to claim 13, wherein the solution containing two or more active ingredients is mixed with the supercritical carbon dioxide by spraying the solution though a nozzle.

19. The process according to claim 13, wherein the solution containing two or more active ingredients is mixed with the supercritical carbon dioxide by pumping the supercritical carbon dioxide into the solution.

20. The process according to claim 13, wherein the supercritical carbon dioxide is mixed with a cosolvent prior to step (b).

21. A process for preparing a pharmaceutical formulation containing a combination of two anti-infective agents comprising:
(a) combining two anti-infective agents with a cosolvent to form a solution;
(b) mixing the solution with supercritical carbon dioxide; and
(c) recovering the precipitate in a dry powder form containing the combination of anti-injective agents.

22. The process according to claim 21, wherein the combination of anti-infective agents is selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

23. The process according to claim 12, wherein the solution is mixed with a supercritical fluid by spraying the solution into a chamber containing the supercritical fluid.

24. The process according to claim 21, wherein the solution is mixed with a supercritical fluid by spraying the solution into a chamber containing the supercritical fluid.

25. A supercritical fluid solution comprising a supercritical fluid and two or more active pharmaceutical ingredients.

26. The supercritical fluid solution according to claim 23, wherein the supercritical fluid is supercritical carbon dioxide.

27. The supercritical fluid solution according to claim 24, wherein two or more of the active pharmaceutical ingredients are anti-infectives.
